(19) United States
(12) Patent Application Publication
(10) Pub. No.: US 2025/0058049 A1
(43) Pub. Date: Feb. 20, 2025

(54) MULTIPLE DELIVERY FLUSH SYRINGE

(71) Applicant: Becton, Dickinson and Company, Franklin Lakes, NJ (US)

(72) Inventors: Praveen Nalawade, Belgaum (IN); Syed Fareed Ahmed, Bangalore (IN)

(73) Assignee: Becton, Dickinson and Company, Franklin Lakes, NJ (US)

(21) Appl. No.: 18/235,098

(22) Filed: Aug. 17, 2023

(51) Int. Cl. A61M 5/31; A61M 5/14; A61M 39/16

(52) U.S. Cl. CPC A61M 5/3134; A61M 5/14; A61M 39/162; A61M 2005/1403; A61M 2005/3104; A61M 2039/1077; A61M 2209/06

(57) ABSTRACT

A flush syringe with a syringe coupler facilitates twice dispensing of intravenous flushing solution to a patient's vascular access device (VAD), with reduced risk of VAD infection. The syringe coupler incorporates a female, first Luer connector on its proximal end that is removably coupled to the male, barrel Luer connector of the syringe, isolating the latter connector from external contamination. The syringe coupler also has a single-use, male, second Luer connector on its distal end of the housing that is adapted for coupled insertion into the patient's VAD, for delivery of at least a portion of the flushing solution retained within the syringe. After the first delivery, the syringe coupler is de-coupled from both the VAD and the syringe. Thereafter, the syringe's unused, sterile, male, barrel Luer connector is directly coupled to the VAD for dispensing the second portion of the flushing solution.

### TECHNICAL FIELD

The present disclosure generally relates to syringes for flushing a vascular access device (VAD) of a patent, such as a patient access port. More particularly, the disclosure relates to a flush syringe that is capable of dispensing flushing solution to a patient VAD in two separate flushing procedures (e.g., before and after drug administration), each using a new, sterile port connector, reducing potential risk of VAD infection.

### BACKGROUND

Vascular access devices (VAD's) are commonly used therapeutic devices and include intravenous (IV) catheters. There are two general classifications of VAD's, peripheral catheters and central venous catheters. Several types of access hubs, ports or valves are coupled to a VAD when delivering a fluid or pharmaceutical. Some IV patient ports are activated by the frusto-conically shaped tip (e.g., a Luer connector) of a syringe barrel to allow fluid communication between the interior of the syringe and the catheter. A Luer connector is a common way to couple or join syringes, catheters, hubbed needles, IV tubes, etc. to each other. When VADs are used for medical patient care, there are potential risks of occlusions and contamination of components.

If not properly maintained, VADs can become occluded. To ensure VADs are used properly and do not become occluded, standards of practice have been developed. These standards include a cleaning procedure, which is commonly referred to as a flush procedure or flushing a catheter. VAD standards of practice usually recommend flush procedures be performed after catheter placement, before fluid infusion, and before and after drug administration, blood sampling, transfusions, and parenteral nutrition. The goal of these flush procedures is to confirm catheter patency, avoid drug incompatibilities, ensure the complete drug dose administration, prevent thrombus formation, and minimize the risk of blood stream infections. Flush procedures require diverse types and amounts of flush solutions. Commonly used flush solutions are saline and/or heparin lock solution. The type of flush solution and amount vary depending on the specific type of catheter. Flush solution volumes between 5 and 10 ml are most common but can range from 1 ml to 20 ml.

Flushing is also performed after medication administration, as residual medicine may remain in the luer portion of the drug delivery syringe and in the catheter. Without a subsequent flushing procedure, the full medication dosage may not be delivered to the patient. Some medications are administration time sensitive and should not remain in the catheter until a subsequent medicine administration flushes the residual quantity of the prior medication through the line. Therefore, sequential flushing with a second flush syringe is performed to resolve the residual medication delivery issue. Medication delivery and companion flushing through VAD delivery systems also inherently expose the delivery system to potential contamination.

Bacteria and other microorganisms may gain entry into a patient's vascular system from diverse types of patient connectors, including by way of non-limiting example, access hubs, ports or valves, upon their connection to the VAD when delivering a fluid or pharmaceutical. Each patient connector, whether an access hub, port, valve or other type of connection, is associated with some risk of transmitting a catheter related bloodstream infection (CRBSI), which can be costly and potentially lethal. In order to decrease CRBSI cases and to ensure VAD's are used and maintained correctly, standards of practice have been developed, which include disinfecting and cleaning procedures. Disinfection caps have been added to the Society for Healthcare Epidemiology of America (SHEA) guidelines and caps are also incorporated into the Infusion Nurses Standards (INS) guidelines.

In developed markets, when utilizing an IV catheter, a needleless patient connector will typically be used to close off the system and then subsequently be accessed to administer medication or other necessary fluids via the catheter to the patient. INS Standards of Practice recommend the use of a needleless connector and state that it should be “consistently and thoroughly disinfected using alcohol, tincture of iodine or chlorhexidine gluconate/alcohol combination prior to each access.” The disinfection of the needleless connector is ultimately intended to aid in the reduction of bacteria that could be living on the surface and possibly lead to a variety of catheter related complications including CRBSI. Nurses will typically utilize a 70% isopropyl alcohol (IPA) pad to complete this disinfection task by doing what is known as “scrubbing the hub.” Currently many nursing units mandate the practice of scrubbing the patient's IV connector hub, even if the connector is presently coupled to an existing disinfection cap.

Typically, four scrubbing procedures (often performed with disinfection caps) are required to administer a single drug through a VAD system in a Saline-Administration of drug-Saline (SAS) workflow process. The first disinfection scrub prepares the patient's VAD connector for drug delivery and receipt of a first flushing syringe to dissolve potential occlusions within the VAD delivery system and its IV catheter. The first flushing syringe is removed after completion of the pre-flush. Thereafter a second disinfection scrub prepares the catheter hub for connection to a medication delivery syringe. A third disinfection scrub prepares the catheter hub for connection of a second flushing syringe to complete a post-medication delivery flush. Lastly, after removal of the second flushing syringe, a fourth disinfection scrub is performed, followed by sealing of the catheter hub with a cap or a locking syringe. In the case of a or Saline-Administration of drug-Saline-Heparin (SASH) workflow process, a third, heparin flushing procedure is required after the second saline flush, which requires a third flush syringe and a fifth disinfection scrub. When more than one drug is administered to a patient, during the workflow process, each additional drug dose is followed by hub disinfection before initiating the next dispensing task.

Therefore, throughout the sequence of procedures necessary to prevent occlusions within VAD systems and to administer medication to a patient there are contamination and microorganism transmission risks that can cause a CRBSI every time a syringe is connected or disconnected from the patient's VAD connector. Best IV catheter contamination practices prohibit re-use of a syringe connector to a patient port connector.

Returning now to procedures for preparing a patient port for drug administration, after the initial IV port scrubbing, followed by flushing with a first, sterile, single-use flush syringe, the practitioner again scrubs the IV port and administers a dosage of medication fluid with a new, sterile, single-use drug syringe, followed by a post-administration scrubbing and flushing with a second, sterile, single-use flush syringe. The initial IV port flush, administration of intravenous medication, and the second IV flush following medication administration typically utilizes three separate, single-use syringes in clinical practice. There is risk that practitioners might re-use a contaminated, single use flushing syringe to perform the pre- and the post-medication delivery flushing during medication administration. Re-use of a flush syringe that was previously connected to a patient IV port increases the risk of transmitting a catheter related bloodstream infection (CRBSI) to the patient.

However, it is appreciated that reducing the number of syringes necessary to flush and administer drugs through a patient's IV port, while conforming to CRBSI reduction practices is beneficial in many ways. Syringe use reduction reduces clinician time and effort necessary to open a fewer number of sterile syringe packages; it also reduces syringe stocking inventory and associated purchase and medical waste disposal costs.

### SUMMARY

A flush syringe incorporates a syringe coupler that facilitates dispensing of intravenous flushing solution to a vascular access device (VAD) of a patient two times (e.g., before and after drug administration), with reduced risk of VAD infection. The syringe embodiments of the present disclosure connect an unused, sterile Luer connector for each of the two flushing procedures. The distal axial end of the syringe coupler provides the first, sterilized Luer connector for coupling to a corresponding hub Luer connector of the VAD and the distal end of the syringe coupler establishes a sterile, fluid seal with the corresponding barrel Luer connector of the syringe barrel.

When the syringe coupler is removed/de-coupled from the syringe's still sterile, barrel Luer connector, the latter is directly coupled to a now re-disinfected hub Luer connector of the VAD. More specifically, the syringe coupler has a female, first Luer connector on its proximal end that is removably coupled to the male, barrel Luer connector of the syringe, which isolates the latter and flushing solution within the syringe barrel from environmental contamination. A male, second Luer connector on its distal end of the housing that is adapted for coupled insertion into the patient's VAD, for delivery of at least a portion of the flushing solution retained within the syringe. Typically, after the first VAD flushing procedure, a clinician disinfects the VAD and administers one or more drugs to the patient through the VAD, disinfecting the VAD after each new drug administration. After the first flushing procedure, the syringe coupler is de-coupled from both the VAD and the male, barrel Luer connector of the syringe. Thereafter, the unused, sterile barrel Luer connector is coupled directly to the VAD for dispensing the second portion of the flushing solution. Each VAD connection by the syringe is done with a fresh, sterile connector. No VAD connector of the syringe is used twice. Thus, a single IV flush syringe can be used to perform both the pre- and post-medication flushing of a patient's IV port. This can lead to a fifty-percent reduction in the number of flush syringes needed to administer drugs to a patient, while still adhering to best CRBSI reduction practices. The syringe use reduction also reduces associated syringe inventory and medical waste disposal costs.

One aspect of the present disclosure pertains to flush syringes for intravenous fluid administration to a patient. In some embodiments, the syringe is pre-filled with flushing solution and packed in sterile packaging. The syringe includes a syringe barrel with a distal end, an open proximal end, and a syringe cavity within the barrel. A syringe plunger is translatable within the syringe cavity. A male, barrel Luer connector is oriented on the distal end of the syringe barrel; it has a male, barrel Luer tip defining an outlet lumen in communication with the syringe cavity. A syringe coupler, coupled to the syringe, includes a housing having proximal and distal axial ends, with a female, first Luer connector on the proximal end of the housing, having a first lumen defined therein, and a male, second Luer connector with a male Luer tip thereof having a second lumen defined therein, on the distal end of the housing. The first passage, formed in the housing, is in fluid communication with the first and second lumens. A hollow sleeve is retained within the first passage. A proximal axial end of the hollow sleeve projects out of the first passage and the first lumen towards the proximal axial end of the housing. The female, first Luer connector of the housing is coupled to male, barrel Luer connector, with the proximal axial end of the hollow sleeve inserted within the outlet of the male, barrel Luer tip, for delivery of the flushing solution retained within the syringe cavity through the hollow sleeve, into the second lumen of the housing's male, second Luer connector. The male, second Luer connector of the housing is adapted for coupled insertion into a corresponding needle-free IV connector of a patient or other type of VAD, for delivery of at least a portion of the flushing solution retained within the syringe cavity into the IV connector. The syringe coupler is selectively removeable from the male, barrel Luer connector, thereby allowing direct coupling of the latter to the corresponding needle-free IV connector for delivery of additional flushing solution retained within the syringe cavity into the IV connector. In one or more embodiments, one or more syringe coupler(s) may be provided and a single flush syringe may be used for multiple fresh tip connections to flush the line between various subsequent drug deliveries.

Another aspect of the disclosure pertains to a method for manufacturing the aforementioned flush syringe. During such syringe manufacture, a syringe barrel is provided with a male, barrel Luer connector oriented on a distal end of the syringe barrel, an open proximal end, and a syringe cavity within the barrel. The syringe's barrel Luer connector has a male, barrel Luer tip defining an outlet lumen in communication with the syringe cavity. A syringe coupler is provided, which includes housing having proximal and distal axial ends. A female, first Luer connector is oriented on the proximal end of the housing and has a first lumen defined therein. A male, second Luer connector is oriented on the distal end of the housing and has a male Luer tip with a second lumen defined therein. The first passage is formed in the housing and is in fluid communication with the first and second lumens. During fabrication of the syringe coupler housing a hollow sleeve is inserted into the first passage, with a proximal axial end thereof projecting out of the first passage and the first lumen towards the proximal axial end of the housing. The female, first Luer connector is coupled to the barrel Luer connector, with the proximal axial end of the hollow sleeve inserted within the outlet of the male, barrel Luer tip. In some embodiments, a selectively removeable storage cap is coupled to the male, second Luer connector of the syringe coupler. In some embodiments, during manufacture of the syringe, the syringe cavity is prefilled with a flushing solution. Thereafter, a translatable syringe plunger is inserted into the syringe barrel within the syringe cavity.

Yet other aspects of this disclosure pertain to a method flushing a patient port during intravenous fluid administration to a patient by using the aforementioned and described flush syringe. When performing this flushing method, it is contemplated that the administering clinician will adhere to approved medical protocols for reducing likelihood of VAD contamination. The clinician disinfects the hub of a patient port having a hub Luer connector or other VAD. The male, second Luer connector of the syringe coupler is coupled to the hub Luer connector of the patient port. By depressing the syringe plunger, a first portion of pre-filled flushing solution retained within the syringe cavity is dispensed into the patient port, through the male, second Luer connector of the syringe coupler. The male, second Luer connector is de-coupled from the hub Luer connector and the hub of the patient port is disinfected. Thereafter, one or more drugs are administered through the hub with drug dosing syringes, disinfecting the hub after delivery of each drug. The syringe coupler is de-coupled from the syringe by decoupling the female, first Luer connector, including its retained hollow sleeve, from the male, barrel Luer connector of the syringe. Thereafter, the syringe coupler and its retained hollow sleeve are discarded. In order to prepare for the second VAD flushing procedure, the hub of the patient port is disinfected and the male, barrel Luer connector is coupled to the hub Luer connector of the patient port and a second portion of the pre-filled flushing solution retained with the syringe cavity is dispensed through the male, barrel Luer connector into the patient port by depressing the syringe plunger. The male, barrel Luer connector is thereafter de-coupled from the hub Luer connector of the patient port, and the syringe is discarded. The hub of the patient port is disinfected again and capped with a sterilized hub cap.

In some embodiments, when practicing the aforementioned patient port flushing method, the flush syringe is removed from sterile packaging and a storage cap is decoupled from the male, second Luer connector of the syringe coupler, prior to coupling latter to the hub Luer connector of the VAD. In other embodiments, the method for flushing a patient port further includes initially disinfecting the Luer hub of the patient port with a scrubbing cap assembly that is pre-coupled to the syringe's storage cap, within the sterile packaging of the flush syringe, prior to coupling the male, second Luer connector of the syringe coupler to the hub Luer connector.

Another aspect of the disclosure pertains to a kit including one or more syringe couplers and a single flush syringe to be used for multiple fresh tip connections to flush a line between multiple or subsequent drug deliveries.

### BRIEF DESCRIPTION OF THE DRAWINGS

Exemplary embodiments of the disclosure are further described in the following detailed description in conjunction with the accompanying drawings, in which:

FIG. 1 is an isometric view of an embodiment of a flush syringe of the present disclosure and an exemplary needle free connector (NFC) patient port type of VAD;

FIG. 2 is a partially exploded view of the syringe of FIG. 1;

FIG. 3 is an isometric view of the syringe and patient port of FIG. 1 coupled together, for a first delivery of flushing solution to the VAD;

FIG. 3A is an enlarged, axial cross-sectional view of the coupled syringe coupler/syringe and the patient port of FIG. 3;

FIG. 4 is an isometric view of the syringe of FIG. 3 after the flushing the patient port with a portion of the flushing solution previously contained within the syringe, and decoupling of the syringe coupler from both the patient port and the syringe barrel;

FIG. 5 is an isometric view of the syringe of FIG. 3, after coupling its barrel Luer connector directly to the patient port, for flushing the patient port a second time with residual flushing solution remaining in the syringe after the first flushing procedure;

FIG. 5A is an enlarged, axial cross-sectional view of the coupled barrel Luer connector of the syringe and patient port of FIG. 5;

FIG. 6 is a partial isometric elevational view of another embodiment of a flush syringe of the present disclosure;

FIG. 7 is a partial exploded view of the flush syringe of FIG. 6;

FIG. 8 is an isometric view of the syringe of FIG. 6 and a patient port coupled together, for a first delivery of flushing solution to the VAD;

FIG. 8A is an enlarged, axial cross-sectional view of the coupled syringe coupler/syringe and the patient port of FIG. 8;

FIG. 9 is an isometric view of the syringe of FIG. 8 after the flushing the patient port with a portion of the flushing solution previously contained within the syringe, and decoupling of the syringe coupler from both the patient port and the syringe barrel;

FIG. 10 is an isometric view of the syringe of FIG. 8, after coupling its barrel Luer connector directly to the patient port, for flushing the patient port a second time with residual flushing solution remaining in the syringe after the first flushing procedure;

FIG. 10A is an enlarged, axial cross-sectional view of the coupled barrel Luer connector of the syringe and patient port of FIG. 10;

FIG. 11 an isometric view of another embodiment of a flush syringe of the present disclosure, which incorporates a scrubbing cap, removed from sterile packaging 11, and an exemplary needle free connector (NFC) patient port type of VAD; and

FIG. 12 is a partial exploded elevational view of the flush syringe of FIG. 11.

To facilitate understanding, identical reference numerals have been used, where possible, to designate identical elements that are common to the figures. The figures are not drawn to scale.

### DETAILED DESCRIPTION

Aspects of embodiments of the flush syringes disclosed herein facilitate two separate, discharges and deliveries of intravenous flushing solution contained within a flush syringe to a vascular access device (VAD) of a patient, with reduced risk of VAD contamination. Each of the two separate syringe connections to the VAD is done with a new, sterile VAD connector. Two separate discharges of uncontaminated flushing solution into a patient's VAD, before and after medication dosing, is possible because the flush syringes disclosed herein have two separate, sterilized Luer connectors available for coupling to the VAD's hub connector. In this way, a clinician has the ability first to flush a patient's VAD through a male, Luer connector on a distal end of a syringe coupler that is oriented between and in fluid communication with both the VAD and the flushing solution contained within the flush syringe. The syringe coupler has a female, Luer connector on its proximal end that is removably coupled to the male, barrel Luer connector of the syringe, which isolates the latter and flushing solution within the syringe barrel from environmental contamination. After the initial flushing procedure, the male, Luer connector of the syringe coupler is de-coupled from the patient's VAD. Typically, after the first VAD flushing procedure, a clinician disinfects the VAD and administers one or more drugs to the patient through the VAD, disinfecting the VAD after each new drug administration. After completion of drug administration, the female, Luer connector of the syringe coupler is de-coupled from the uncontaminated, male, barrel Luer connector of the syringe. Thereafter, the uncontaminated barrel Luer connector is coupled directly to the VAD for dispensing the second portion of the flushing solution. In one or more embodiments, one or more syringe coupler(s) may be provided and a single flush syringe may be used for multiple fresh tip connections to flush the line between various subsequent drug deliveries.

In this disclosure, a convention is followed wherein the distal end of the device is the end closest to a patient, e.g., for delivery of one or more drugs to the patient, and the proximal end of the device is the end away from the patient and closest to a clinician or other medical practitioner. With respect to terms used in this disclosure, the following definitions are provided.

As used herein, the use of “a,” “an,” and “the” includes the singular and plural.

As used herein, the term “Luer connector” refers to a connection collar that is the standard way of attaching syringes, catheters, hubbed needles, IV tubes, etc. to each other. The Luer connector consists of male and female interlocking tubes, slightly tapered to hold together better with even just a simple pressure/twist fit. Luer connectors can optionally include an additional outer rim of threading, allowing them to be more secure. The Luer connector male end is associated with a flush syringe and can interlock and connect to the female end located on the vascular access device (VAD). A Luer connector also has a distal end channel that releasably attaches the Luer connector to the hub of a VAD, and a proximal end channel that releasably attaches the Luer connector to the barrel of a syringe.

As used herein, ISO 80369-7:2016 defines a specification for standard Luer connectors including a 6% taper between the distal end and the proximal end. A male standard Luer connector increases from the open distal end to the proximal end. A female standard Luer connector decreases from the open proximal end to the distal end. According to ISO 80369-7:2016, a male standard Luer connector has an outer cross-sectional diameter measured 0.75 mm from the distal end of the tip of between 3.970 mm and 4.072 mm. The length of the male standard Luer taper is between 7.500 mm to 10.500 mm. The outer cross-sectional diameter measured 7.500 mm from the distal end of the tip is between 4.376 mm and 4.476 mm. As used herein, the phrases “male standard Luer connector” and “female standard Luer connector” shall refer to connectors having the dimensions described in ISO 80369-7, which is hereby incorporated by reference in its entirety.

As would be readily appreciated by skilled artisans in the relevant art, while descriptive terms such as “tip”, “hub”, “thread”, “protrusion/insert”, “tab”, “slope”, “wall”, “top”, “side”, “bottom” and others are used throughout this specification to facilitate understanding, it is not intended to limit any components that can be used in combinations or individually or to require specific spatial orientations, to implement various aspects of the embodiments of the present disclosure.

Before describing several exemplary embodiments of the disclosure, it is to be understood that the disclosure is not limited to the details of construction or process steps set forth in the following description. The disclosure is capable of other embodiments and of being practiced or being conducted in many ways.

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

In an exemplary implementation of the embodiments of present disclosure, a barrel of a syringe includes a distal end having a needleless connection. In one or more embodiments, the needleless connection includes at least one thread and other features in all combinations allowing it to interface with a corresponding thread or plurality of threads of a corresponding connector.

According to further exemplary implementations of the embodiments of the present disclosure, configuration of structural elements making up the needleless connector include a collar protruding from the distal end of the barrel, the collar comprising at least one thread to connect to the corresponding thread or plurality of threads of a corresponding connector.

According to still further exemplary implementations of the embodiments of the present disclosure, the collar or the needleless connector bends or elastically deforms to allow better interference fit compliance with corresponding connectors.

According to still further exemplary implementations of the embodiments of the present disclosure, the needleless connector may comprise female threads that are sized and have a thread pattern that will engage with a standard ISO594-2 type of male fitting and/or male threads that are sized and have a thread pattern that will engage with a standard ISO594-2 type of female fitting. An example of an ISO594-2 type of fitting is a Q-style fitting.

In one or more embodiments, a female connector may be selected from the group consisting essentially of: needle-type connectors (for direct injection into a patient or insertion into a drug vial for aspiration of a drug dose therefrom), needle-free connectors, catheter Luer connectors, stopcocks, and hemodialysis connectors. In one or more embodiments, the needleless connector is selected from a Q-Syte connector, MaxPlus, MaxPlus Clear, MaxZero, UltraSite, Caresite, InVision-Plus, Safeline, OneLink, V-Link, ClearLink, NeutraClear, Clave, MicroClave, MicroClave Clear, Neutron, NanoClave, Kendall, Nexus, InVision, Vadsite, Bionector, etc.

In one or more embodiments, the male connector may be an intravenous tubing end or a stopcock.

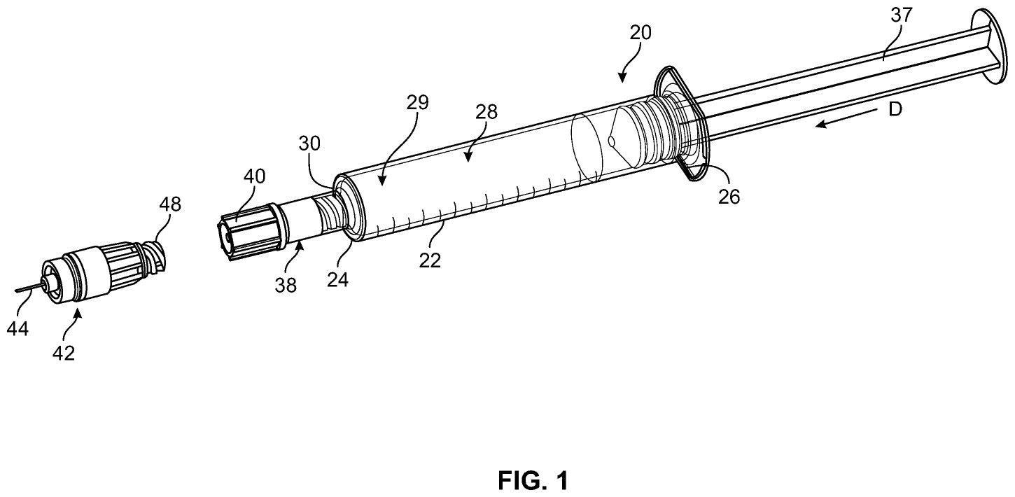

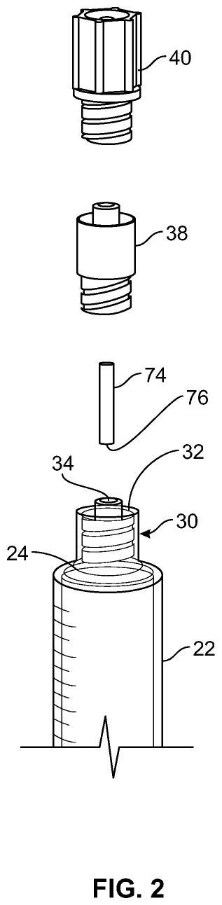

Referring now to the drawings, the first aspect of the present disclosure is shown in FIGS. 1-5A, wherein a flush syringe comprises a syringe barrel 22, which defines a distal end 24 an open, proximal end 26, and an internal syringe or barrel cavity 28. In some embodiments, the syringe cavity 28 is pre-filled with a flushing solution 29 during syringe manufacture. In some embodiments, the volume of flushing solution 29 that is retained within the syringe cavity 28 is 10 ml. The conical profile, distal end 24 of the barrel incorporates a threaded, male, barrel Luer connector 30, which includes a male, barrel Luer tip 32. An outlet lumen 34, formed in the barrel Luer tip 32 is in fluid communication with the syringe cavity 28. The threaded, male, barrel Luer connector 30 has a collar 35 with at least one internally directed thread 36 that circumscribe the barrel Luer tip 32. A syringe plunger 37 advances in the direction of the arrow D, to dispense flushing solution under pressure from the syringe cavity 28 through the outlet lumen of the barrel Luer connector 30. In some embodiments, as shown in FIG. 1, the syringe 20 is pre-filled with flushing solution 29, with a proximal axial end of the syringe coupler 38 coupled to the barrel Luer connector 30, and a storage or end cap 40 coupled to a distal end of the syringe coupler. Typically, the assembled, pre-filled, syringe 20, syringe coupler 38 and end cap 40 are prepackaged in sterile packaging (not shown). An exemplary flush syringe is shown and described in United States Utility U.S. Pat. No. 8,632,521B2, entitled “Syringe Assembly”, the entire contents of which is hereby incorporated by reference in all jurisdictions whose patent laws permit such incorporation.

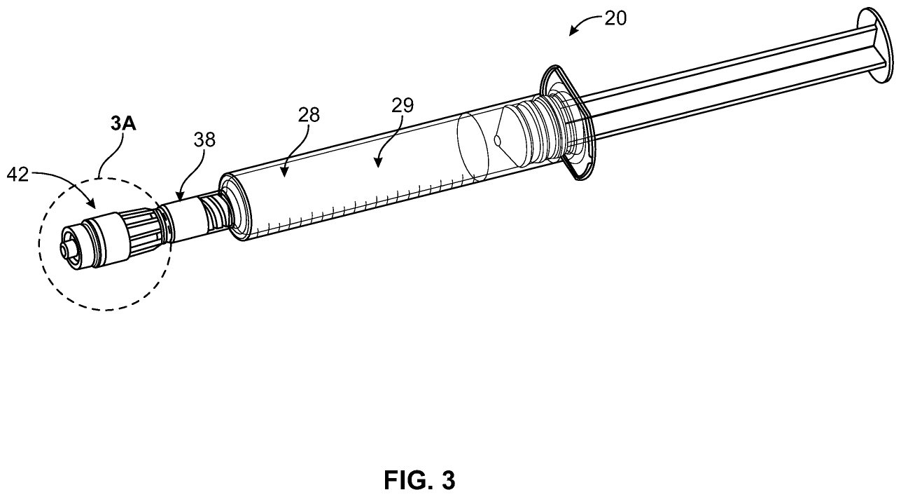

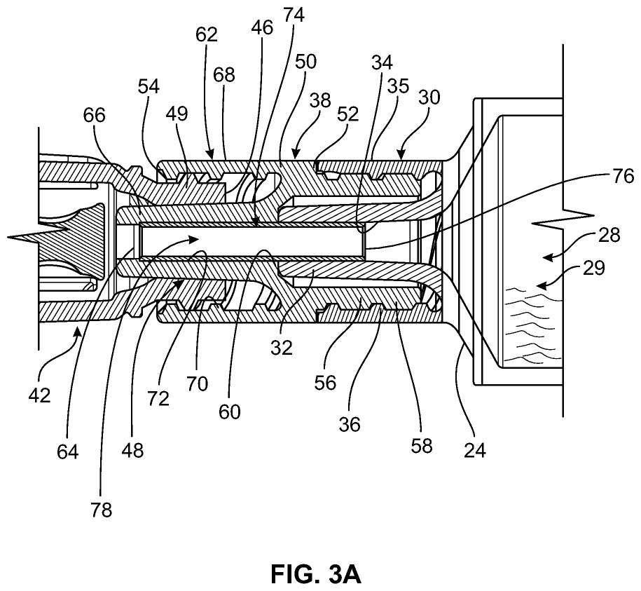

FIGS. 1, 3 and 3A show a typical needle free connection (NFC)-type, IV port 42, having a patient IV catheter 44 and a threaded, female, hub Luer connector 48 with external threads 49. Referring more specifically to the enlargement FIG. 3A, the syringe coupler 38 comprises a housing 50, with proximal 52 and distal 54 axial ends. A threaded, female, first Luer connector 56 on the proximal axial end 52 of the housing 50 defines external threads 58 that are in mating engagement with the corresponding internally facing threads 36 formed within the collar 35 of the male, barrel Luer connector 30. A first lumen 60 of the female, first Luer connector 56 is in fluid communication with the outlet lumen 34 of the barrel Luer connector 30. A threaded, male, second Luer connector 62 on the distal axial end 54 of the housing 50 has a second lumen 64 formed within the male Luer tip 66, a collar 68 that defines at least one internally directed thread 70. The male, second Luer connector 62 and the barrel Luer connector 30 have compatible construction; either of these male connectors intentionally is capable of coupling to a corresponding hub Luer connector 48 of the IV port 42.

The housing defines a first passage 72 that is in fluid communication with the first 60 and second 64 lumens. A hollow sleeve 74 is retained within the first passage 72. In some embodiments, the hollow sleeve 74 is bonded within the first passage 72 to the housing 50. A proximal axial end 76 of the hollow sleeve 74 projects out of the first passage 72 and the first lumen 60 towards the proximal axial end 52 of the housing 50. The proximal axial end 76 of the hollow sleeve 74 is inserted within the outlet lumen 34 of the male, barrel Luer tip 32. In some embodiments, insertion of the hollow sleeve 74 into the outlet lumen 34 effectively isolates the outlet of the barrel Luer connector and the syringe cavity 28 from external contamination. By insertion of the proximal axial end 76 of the hollow sleeve 74 into the outlet lumen 34, a flow channel or pathway is created for delivery of the flushing solution 29 retained within the syringe cavity 28, through a hollow sleeve lumen 78 of the hollow sleeve, into the second lumen 64 of the housing's male, second Luer connector 62. In turn, at least a portion of the flushing solution 29 is dispensed into the hub Luer connector 48 from the mutually coupled, male, second Luer connector 62 of the syringe coupler 38 upon advancement of the syringe plunger 37. Typically, about 5 ml of flushing solution is dispensed into the hub Luer connector 48, which is half of the syringe's 10 ml capacity. In some instances, more or less than 5 ml of flushing solution is dispensed into the hub Luer connector 48, which is half of the syringe's 10 ml capacity.

After the initial flushing of the patient's IV port 42 is completed, the male, second Luer connector 62 of the syringe coupler 38 is decoupled from the IV port 42. The syringe coupler 38 remains coupled the barrel Luer connector 30 of the syringe 20, and continues to isolate the latter, along with the typically remaining 5 ml of flushing solution 29 in the syringe cavity 28, from the external environment. After mutually de-coupling the syringe coupler 38 and the IV port 42, a clinician disinfects the IV port 42 and dispenses one or more drugs or other medicines to the patient, with respective dosing syringes (not shown). Under recognized drug administration protocols, the IV port 42 is flushed again. The flush syringe embodiments of the present disclosure allow the clinician to re-flush the IV port 42 with the rest of the flushing solution 29 remaining in the same syringe 20.

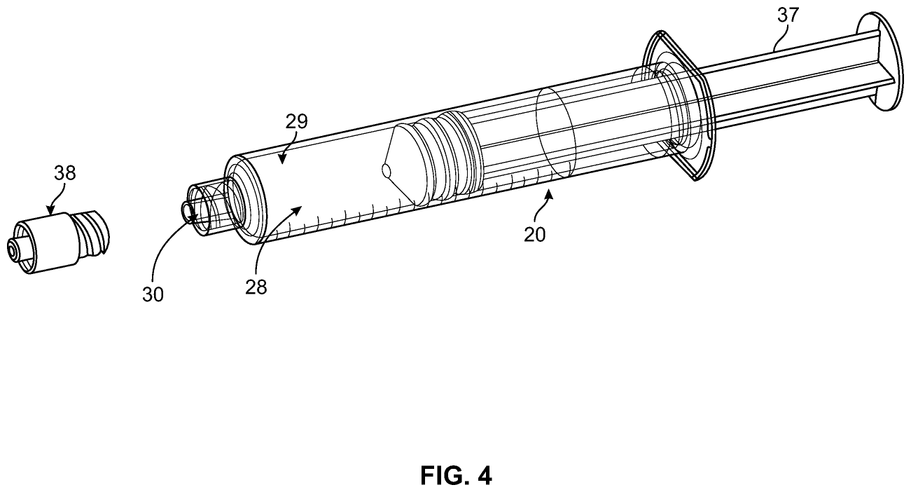

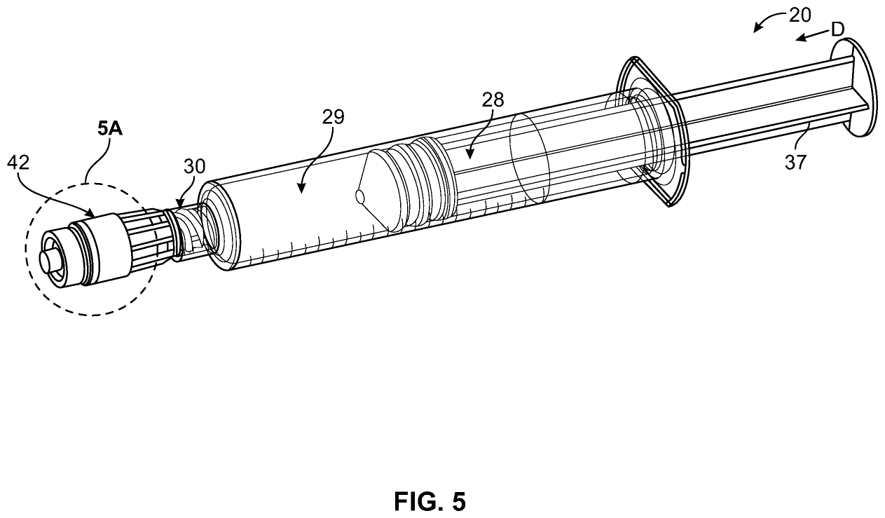

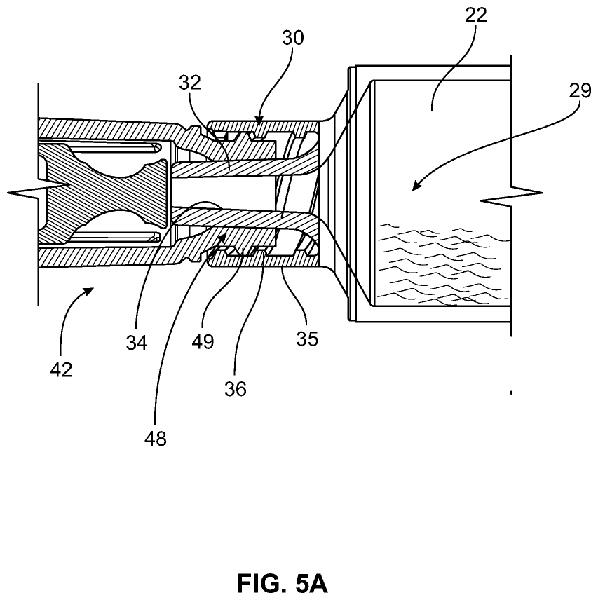

A shown in FIG. 4, to prepare the syringe 20 for a second flush of the patient IV port, the syringe coupler 38, which up to now has isolated the barrel Luer connector 30 of the syringe 20 from the external environment, is de-coupled from the barrel Luer connector 30 of the syringe 20. In FIG. 5 and the FIG. 5A enlargement, the barrel Luer connector 30 of the syringe 20 is coupled directly to the IV port 42, comparable to the prior coupling of the male, second Luer connector 62 of the syringe coupler 38 to the same IV port. Namely, threads 36 of the collar 35 mutually engage the corresponding threads of the hub Luer connector 48. Advancement of the syringe plunger 37 (arrow D) discharges the typical remaining, approximately 5 ml of flushing solution 29 from the syringe cavity 28 into the IV port 42, via the outlet lumen 34. Upon completion of the second flush, the syringe 20 is decoupled from the IV port 42 and discarded. The clinician completes remaining drug administration protocols, including disinfecting the IV port and filling same with a heparin solution, re-disinfecting and capping the IV port.

In one or more embodiments, one or more syringe coupler(s) may be provided and a single flush syringe may be used for multiple fresh tip connections to flush the line between various subsequent drug deliveries.

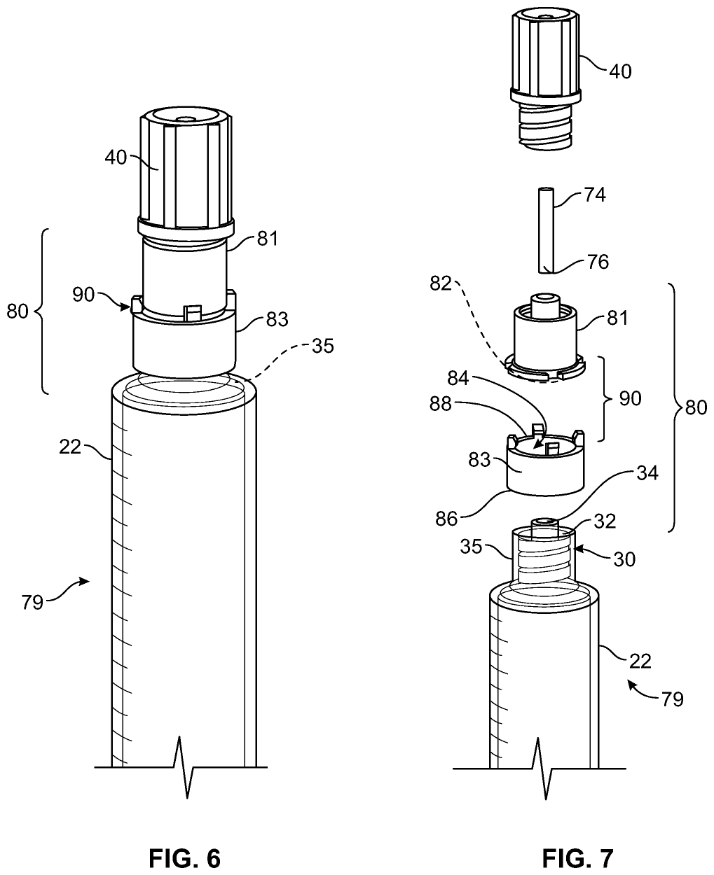

Another aspect of the disclosure is an alternative flush syringe embodiment 79 of FIGS. 6-10A, wherein a syringe coupler 80 is coupled to the outer circumferential surface of the collar 35 of barrel Luer connector 30. The syringe barrel 22 and its barrel Luer connector 30, the syringe plunger 37, and the end cap 40 are identical to those of the previously described syringe 20 of FIGS. 1-5A.

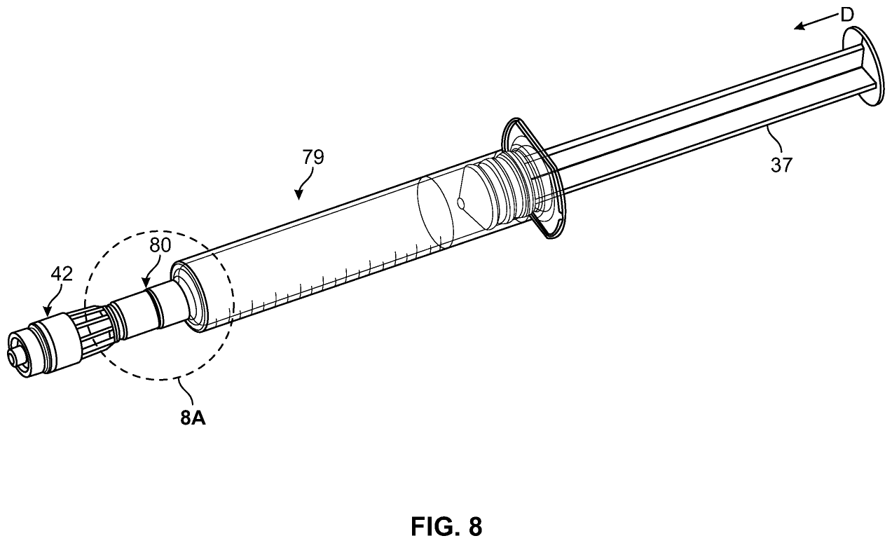

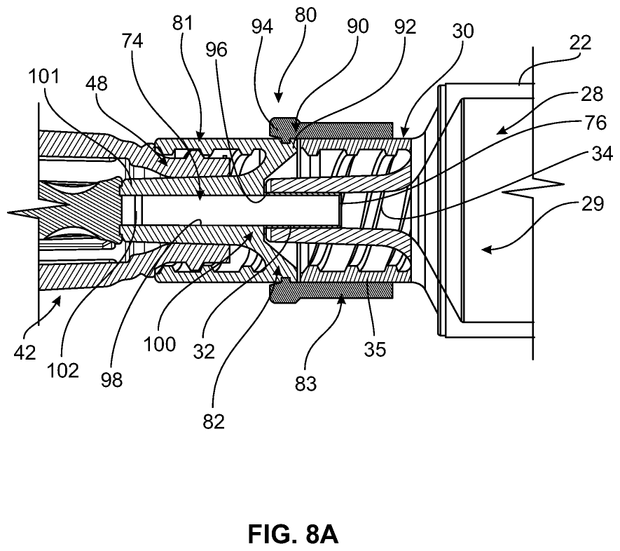

As shown in FIGS. 7 and 8A, the syringe 79 includes a syringe coupler 80 comprising a housing 81. The proximal axial end of the housing 81 includes a female, first Luer connector 82, for mating with the male, barrel Luer connector 30. The proximal axial end of the syringe coupler 80 further comprises a coupling sleeve 83 that defines an internal coupling cavity 84. The proximal axial end 86 of the coupling sleeve 83 receives and is coupled with the exterior circumferential surface of the threaded collar 35 of the corresponding male, barrel Luer connector 30. The distal axial end 88 of the coupling sleeve 83 is selectively joined with and coupled to the proximal, axial end of the housing 81 by a selectively engageable snap fitting 90. Upon locking engagement of the snap fitting 90, it prevents axial separation of the coupling sleeve 83 and the corresponding male, barrel Luer connector 30. The snap fitting comprises a circumferential flange 92 on the housing 81 and opposing, mating, biased hooks 94 on the distal axial end 88 of the coupling sleeve 83. Snap-fit engagement is accomplished by axially inserting the biased hooks 94 within gaps in the split circumferential flange 92 and then twisting the housing 81 relative to the coupling sleeve 83. In other embodiments, the circumferential flange is not a split flange; the hooks are snapped over the flange without twisting the housing. In other embodiments, the biased hooks are oriented on the housing and the circumferential flange (whether split or solid) is oriented on the coupling sleeve. In one or more embodiment, the circumferential flange projects radially outward from the housing.

In FIGS. 8 and 8A, the syringe coupler 80 is coupled to an IV port 42 in the same way as the previously described embodiment of the syringe coupler 38 of FIG. 3. Referring more specifically to the enlargement FIG. 8A, the housing 81 incorporates a threaded, female, first Luer connector 82. A first lumen 96 of the female, first Luer connector 82 and an adjoining first passage 98 in the housing 81 are in fluid communication with the outlet lumen 34 of the barrel Luer connector 30. A threaded, male, second Luer connector 100 on the distal end of the housing 81 has a male Luer tip 101 with an internal second lumen 102 formed therein. The male, second Luer connector 100 and the barrel Luer connector 30 have compatible construction; either of these male connectors intentionally is capable of coupling to a corresponding hub Luer connector 48 of the IV port 42. The first passage 98 is in fluid communication with the first 96 and second 102 lumens. A hollow sleeve 74 is retained within the first passage 98. A proximal axial end 76 of the hollow sleeve 74 projects out of the first passage 98 and the first lumen 96 towards the proximal axial end of the housing 81. The proximal axial end 76 of the hollow sleeve 74 is inserted within the outlet lumen 34 of the male, barrel Luer tip 32, creating a flow channel or pathway for delivery of the flushing solution 29 retained within the syringe cavity 28, through a hollow sleeve lumen 78 of the hollow sleeve, into the second lumen 102 of the housing's male, second Luer connector 100. In turn, at least a portion of the flushing solution 29 is dispensed into the hub Luer connector 48 from the mutually coupled, male, second Luer connector 100 of the syringe coupler 80 upon advancement of the syringe plunger 37.

After the initial flushing of the patient's IV port 42 is completed, the male, second Luer connector 100 of the syringe coupler 80 is decoupled from the IV port 42. The syringe coupler 80 remains coupled the barrel Luer connector 30 of the syringe 79, and continues to isolate the latter, along with the flushing solution 29 in the syringe cavity 28 from the external environment. After mutually de-coupling the syringe coupler 80 and the IV port 42, a clinician disinfects the IV port 42 and dispenses one or more drugs or other medicines to the patient, with respective dosing syringes (not shown). Under recognized drug administration protocols, the IV port 42 is flushed again. The flush syringe embodiments of the present disclosure allow the clinician to re-flush the IV port 42 with the rest of the flushing solution 29 remaining in the same syringe 79.

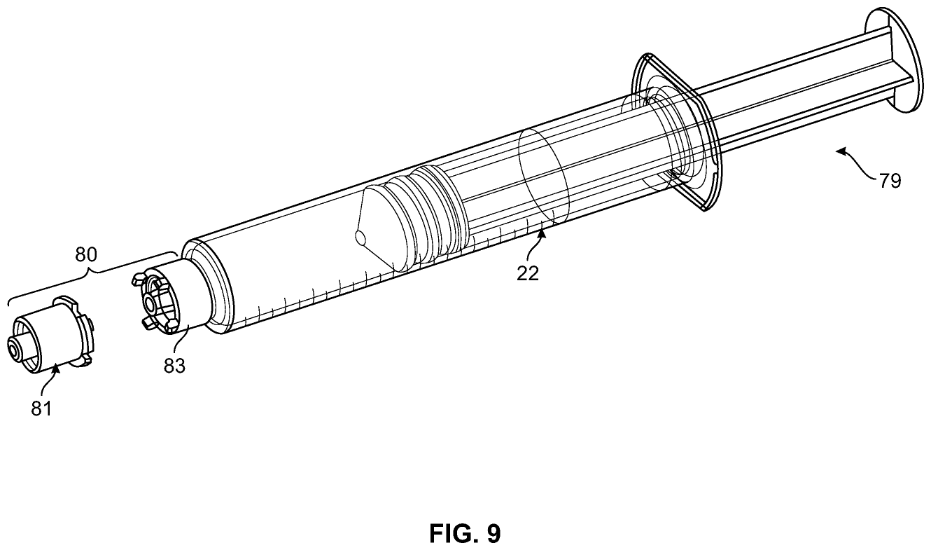

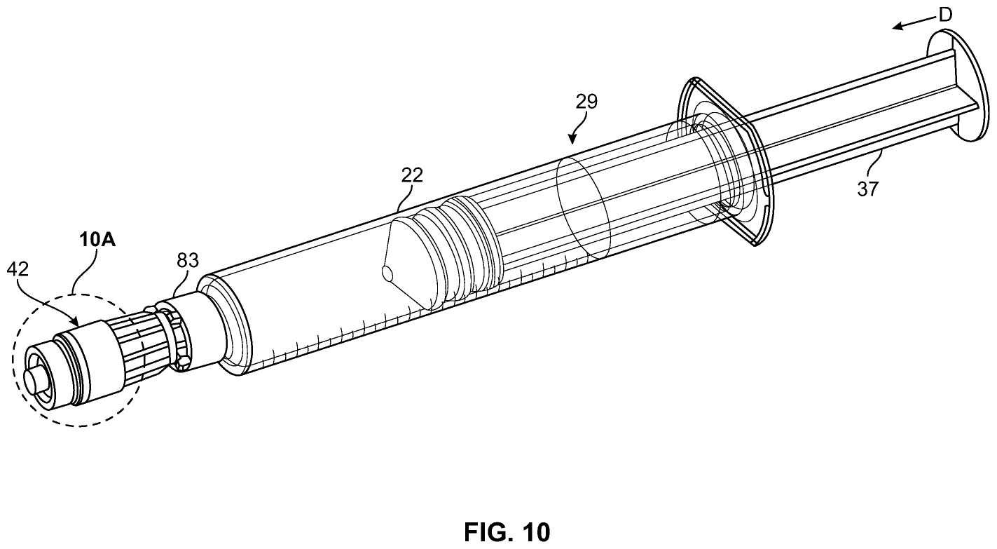

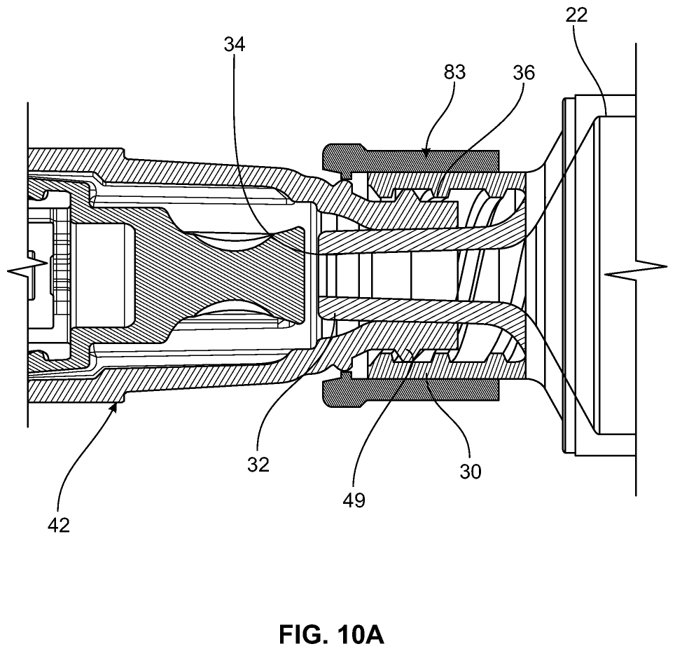

A shown in FIG. 9, to prepare the syringe 79 for a second flush of the patient IV port, the housing 81 of the syringe coupler 80, which up to now has isolated the barrel Luer connector 30 of the syringe 20 from the external environment, is de-coupled from the coupling sleeve 83; the coupling sleeve remains coupled to the barrel Luer connector 30 of the syringe 79. In FIG. 10 and the FIG. 10A enlargement, the barrel Luer connector 30 of the syringe 79 is coupled directly to the IV port 42, comparable to the prior coupling of the male, second Luer connector 100 of the syringe coupler 80 to the same IV port. Namely, threads 36 of the collar 35 mutually engage the corresponding external threads 49 of the hub Luer connector 48. Advancement of the syringe plunger 37 (arrow D) discharges remaining flushing solution 29 from the syringe cavity 28 into the IV port 42, via the outlet lumen 34. Upon completion of the second flush, the syringe 79 is decoupled from the IV port 42 and discarded. The clinician completes remaining drug administration protocols, including disinfecting the IV port and filling same with a heparin solution, re-disinfecting and capping the IV port.

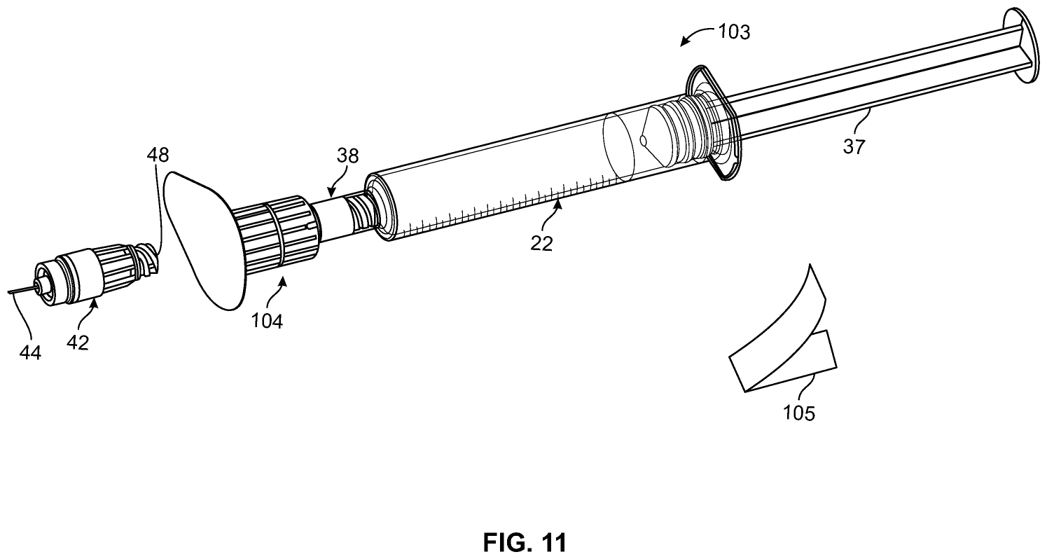

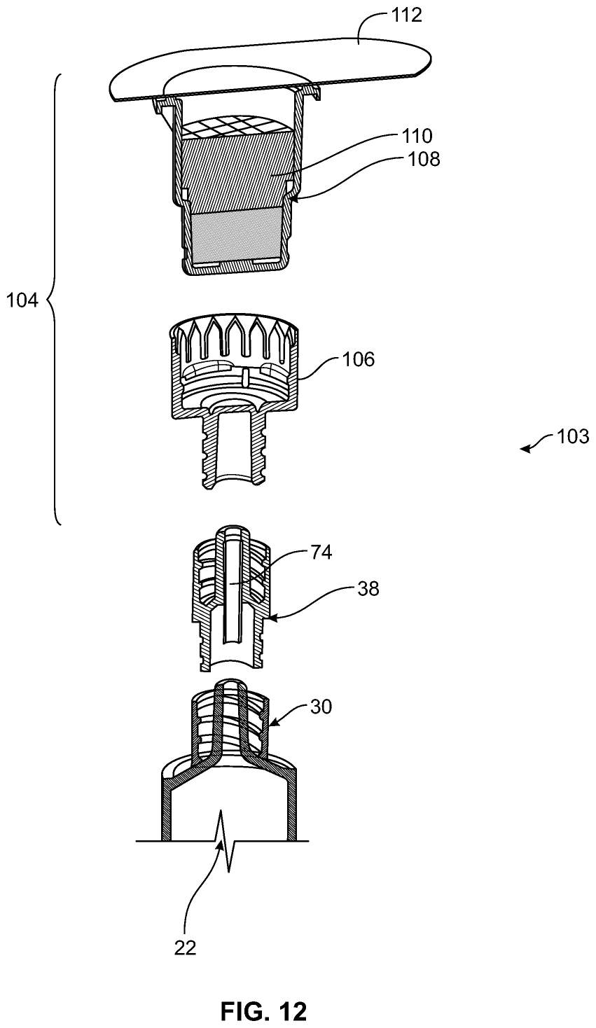

In accordance with another aspect of the disclosure, another embodiment of a flush syringe 103 is shown in FIGS. 11 and 12, for coupling to a hub Luer connector 48 of a patient IV port 42 and associated IV catheter 44. The syringe barrel 22, syringe plunger 37 and syringe coupler 38 of the flush syringe 103 are identical to those of the previously described syringe embodiment 20 of FIGS. 1-5A. The flush syringe 103 further comprises a scrubbing cap assembly 104. In FIG. 11, the fully assembled, pre-filled flush syringe 103 is removed from its sterile packaging 105, to prepare it for initial disinfection and flushing of the patient's IV port 42. The scrubbing cap assembly 104 comprises storage cap 106 that is coupled to the syringe coupler 38 during syringe 103 shipment, and a scrub cap 108. A scrub and disinfection pad 110, impregnated with an antimicrobial agent, is oriented within an internal cavity of the scrub cap 108. Peel lid 112 covers the internal cavity of the scrub cap. Prior to initializing the first port flush, the scrubbing cap assembly 104 is removed from the syringe coupler 38. In some embodiments, scrub cap 108 is used to disinfect the IV port 42 prior to coupling the syringe coupler to the IV port and initializing the first port flush. In other embodiments, scrub cap 108 is reserved to disinfect the patient IV port later in the drug administration sequence. An exemplary scrubbing cap assembly is shown and described in United States Utility U.S. Pat. No. 11,464,961B2, titled “Needleless Connector and Access Port Disinfection Cleaner and Antimicrobial Protection Cap”, the entire contents of which is hereby incorporated by reference in all jurisdictions whose patent laws permit such incorporation.

In some embodiments, the scrub/disinfection pad 110 compresses upon contact with the patient IV port 42. In some embodiments, the scrub/disinfection pad 110 compresses upon contact with the patient connector 46. In some embodiments, the scrub/disinfection pad 110 further comprises an antimicrobial material and/or is impregnated with an antimicrobial agent. More particularly, in some embodiments, the scrub/disinfection pad 110 is a disinfectant sponge or other alternative absorbent material retained within the scrub cap 108. In some embodiments, the scrub/disinfection pad 110 includes an absorbed or infiltrated, liquid or gel, disinfectant or an antimicrobial agent for disinfecting the patient connector 46 or other medical device that is to be cleaned with the scrub cap 108. In one or more embodiments, the absorbent material comprising scrub/disinfection pad 110 is a nonwoven material, foam, or a sponge having a porous structure. In a specific embodiment, the foam is a polyurethane foam. In a specific embodiment, the absorbent material is a sponge.

The scrub/disinfection pad 110 retains disinfectant or antimicrobial agent in its porous structure due to surface tension and releases disinfectant or antimicrobial agent when squeezed or compressed by contact with the patient connector 46 or other medical device that is to be cleaned by the scrub cap 108. The scrubbing cap assembly 104 is compatible in interacting with various disinfectants. In one or more embodiments, the disinfectant or antimicrobial agent includes variations of alcohol or chlorhexidine. In one or more embodiments, the disinfectant or antimicrobial agent is selected from the group consisting essentially of isopropyl alcohol, ethanol, 2-propanol, butanol, methylparaben, ethyl paraben, propylparaben, propyl gallate, butylated hydroxy anisole (BHA), butylated hydroxytoluene, t-butyl-hydroquinone, chloroxylenol, chlorhexidine, chlorhexidine diacetate, chlorhexidine gluconate, povidone iodine, alcohol, dichlorobenzyl alcohol, dehydroacetic acid, hexetidine, triclosan, hydrogen peroxide, colloidal silver, benzethonium chloride, benzalkonium chloride, octenidine, antibiotic, and mixtures thereof. In a specific embodiment, the disinfectant or antimicrobial agent comprises at least one of chlorhexidine gluconate and chlorhexidine diacetate. In one or more embodiments, the disinfectant or antimicrobial agent is a fluid or a gel. In one or more specific embodiments, the disinfectant or antimicrobial agent 130 is 70% isopropyl alcohol (IPA).

The peel lid 112 covers the distal open end of the scrub cap 108. In other embodiments, the scrub cap does not include a peel lid covering the distal open end of the inspection cap. In one or more embodiments, the peel lid 112 is a peelable seal, which comprises an aluminum or multi-layer polymer film peel back top. The peel lid 112 minimizes entry of potential particulate hazard and also provides a substantially impermeable enclosure for the open end of the scrub cap 108, provides leak prevention, protects the absorbed, infiltrated contents of the scrub/disinfection pad 110 or other disinfectant media contained within the cap, and/or maintains a sealed, sterilized environment within the scrub cap. Seal composition of the peel lid 112 provides a sufficient environmental seal between its aluminum or polymer film and the distal open end of the scrub cap 108 at a range of temperatures, pressures, and humidity levels expected within a medical treatment facility. In some embodiments, the peel lid 112 is heat-scaled or induction-sealed to the distal open end of the scrub cap 108. In other embodiments, the peel lid 112 is sealed to the distal axial end 88 of the scrub cap 108 with pressure or thermally sensitive adhesive.

Components of the flush syringe embodiments 20, 79 and 103, and the other embodiments described herein, are constructed by injection molding or vacuum thermal forming. Exemplary materials used to construct the syringe coupler 38 or 80, the end cap 40, and the scrubbing cap assembly 104 include thermoplastic polymers. The hollow sleeve 74 is an elastomeric sleeve that is constructed by compression molding of cross-linked rubber or thermoplastic elastomer materials. Generally, the components of the flush syringe embodiments 20, 79 and 103 are constructed with types of medical grade, plastic materials such as polycarbonate, polypropylene, polyethylene, glycol-modified polyethylene terephthalate, acrylonitrile butadiene styrene or any other moldable plastic material used in medical devices.

One or more of the flush syringe embodiments described in this disclosure are manufactured as follows. In one exemplary manufacturing embodiment, referring to the syringe 20 of FIGS. 1-5A, a flush syringe 20 is provided, generally comprising a syringe barrel 22 with a male, barrel Luer connector 30 oriented on a distal end of the syringe barrel, an open proximal end, and a syringe cavity 28 within the barrel. In some embodiments, the barrel Luer connector 30 has a male, barrel Luer tip 32 defining an outlet lumen 34 in communication with the syringe cavity 28. In embodiments, an existing, off-the-shelf, 10 ml capacity flush syringe is utilized for manufacture of the sequential delivery flushing syringe of the present disclosure, as well as an existing, off-the-shelf, end cap 40.

The manufactured syringe 20 embodiment incorporates a syringe coupler 38, which includes the previously described a housing 50 having proximal and distal axial ends; a female, first Luer connector 56 on the proximal end of the housing, having a first lumen 60 defined therein. The syringe coupler 38 further comprises a male, second Luer connector 62 on the distal end of the housing, with a male Luer tip 66 thereof having a second lumen 64. The first passage 72 is formed in the housing, in fluid communication with the first and second lumens. A hollow sleeve 74 is inserted into the first passage 72, with a proximal axial end 76 thereof projecting out of the first passage and the first lumen 60 towards the proximal axial end of the housing 50. The female, first Luer connector 56 is coupled to the barrel Luer connector 30, with the proximal axial end 76 of the hollow sleeve 74 inserted within the outlet lumen 34 of the male, barrel Luer tip32. The storage cap or end cap 40 is coupled to the male, second Luer connector 62 of the syringe coupler 38. The syringe cavity 28 is pre-filled with a flushing solution 29 prior to insertion of the syringe plunger 37 into the syringe barrel 22 within the syringe cavity 28. In some embodiments, the completed, pre-filled flush syringe is sterilized and packaged in a sterile packaging container.

Reference throughout this specification to “one embodiment,” “certain embodiments,” “various embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in various embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments. In addition, it is to be understood that the phraseology and terminology used herein is for the purpose of description and should not be regarded as limiting. The use of “including.” “comprising.” or “having” and variations thereof herein is meant to encompass the items listed thereafter and equivalents thereof as well as additional items. Unless specified or limited otherwise, the terms “mounted.” “connected,” “supported,” and “coupled” and variations thereof are to be interpreted broadly; they encompass direct and indirect mountings, connections, supports, and couplings. Further, “connected” and “coupled” are not restricted to physical, mechanical, or electrical connections or couplings.

Another aspect of the disclosure pertains to a kit including one or more syringe couplers and a single flush syringe to be used for multiple fresh tip connections to flush a line between multiple or subsequent drug deliveries.

Although the disclosure herein provided a description with reference to embodiments, it is to be understood that these embodiments are merely illustrative of the principles and applications of the disclosure. It will be apparent to those skilled in the art that various modifications and variations can be made to the present disclosure without departing from the spirit and scope thereof. For example, the structural features and operation of specific syringe embodiments 20, 79, 103 have been described herein. Various male and female, threaded Luer connectors that have been described are integrated into the syringe barrels 22 as well as the syringe couplers 38, 80. In some embodiments those Luer connectors are not threaded Luer connectors. Similarly, while the snap fitting 90 of the syringe coupler 80 has been described as comprising a circumferential rim 92 and hooks 94 other embodiments utilize other types of snap fittings. Thus, it is intended that the present disclosure include modifications and variations that are within the scope of the appended claims and their equivalents. The appended claims are not limited to the exemplary embodiment details of construction and the arrangement of components set forth in the description or illustrated in the drawings.

What is claimed is:

1. A flush syringe for intravenous fluid administration to a patient, comprising:
a syringe barrel with a distal end, an open proximal end, and a syringe cavity within the syringe barrel;
a syringe plunger translatable within the syringe cavity;
a male, barrel Luer connector oriented on the distal end of the syringe barrel, having a male, barrel Luer tip defining an outlet lumen in communication with the syringe cavity;
a syringe coupler, including:
a housing having proximal and distal axial ends;
a female, first Luer connector on the proximal end of the housing, having a first lumen defined therein;
a male, second Luer connector on the distal end of the housing, a male Luer tip thereof having a second lumen defined therein;
a first passage formed in the housing, in fluid communication with the first and second lumens; and
a hollow sleeve retained within the first passage, a proximal axial end of the hollow sleeve projecting out of the first passage and the first lumen towards the proximal axial end of the housing;
the female, first Luer connector of the housing coupled to male, barrel Luer connector, with the proximal axial end of the hollow sleeve inserted within the outlet of the male, barrel Luer tip, for delivery of a flushing solution retained within the syringe cavity through the hollow sleeve, into the second lumen of the housing's male, second Luer connector;
the male, second Luer connector of the housing adapted for coupled insertion into a corresponding needle-free IV connector of a patient for delivery of at least a portion of the flushing solution retained within the syringe cavity into the IV connector; and
the syringe coupler selectively removeable from the male, barrel Luer connector, thereby allowing direct coupling of the latter to the corresponding needle-free IV connector for delivery of additional flushing solution retained within the syringe cavity into the IV connector.

2. The flush syringe of claim 1, where all of the Luer connectors comprise threaded Luer connectors, the syringe coupler selectively removeable from the flush syringe by unscrewing the female, first Luer connector of the housing from the male, barrel Luer connector.

3. The flush syringe of claim 2, the male, barrel Luer connector and the male, second Luer connector of the syringe coupler further comprising threaded collars circumscribing respective male Luer tips thereof.

4. The flush syringe of claim 3, the housing further comprising a coupling sleeve defining an internal coupling cavity on a proximal axial end thereof for slidable receipt of an exterior circumferential surface of a threaded outer collar of the corresponding male, barrel Luer connector, and a distal axial end of the coupling sleeve coupled to the proximal, axial end of the housing.

5. The flush syringe of claim 4, further comprising the distal axial end of the coupling sleeve and the proximal axial end of the housing selectively joined by a selectively engageable snap fitting, which when engaged, prevents axial separation of the coupling sleeve and the corresponding male, barrel Luer connector.

6. The flush syringe of claim 5, the snap fitting comprising a circumferential flange oriented on the coupling sleeve or the housing and opposing, mating, biased hooks on the other of the coupling sleeve or the housing that engage the circumferential flange and prevent axial separation of the coupling sleeve and the corresponding male, barrel Luer connector when so engaged.

7. The flush syringe of claim 1, further comprising a selectively removeable storage cap coupled to the male, second Luer connector of the syringe coupler.

8. The flush syringe of claim 7, the storage cap further comprising a scrubbing cap assembly that incorporates an antimicrobial agent therein for disinfecting a patient port prior to coupling the male, second Luer connector to the patient port.

9. The flush syringe of claim 2, further comprising a selectively removeable storage cap coupled to the male, second Luer connector of the syringe coupler.

10. The flush syringe of claim 9, the storage cap further comprising a scrubbing cap assembly that incorporates an antimicrobial agent therein for disinfecting a patient port prior to coupling the male, second Luer connector to the patient port.

11. A method for manufacturing the flush syringe of claim 1, comprising:
providing a syringe barrel with a male, barrel Luer connector oriented on a distal end of the syringe barrel, an open proximal end, and a syringe cavity within the barrel, the barrel Luer connector having a male, barrel Luer tip defining an outlet lumen in communication with the syringe cavity;
providing a syringe coupler, including: a housing having proximal and distal axial ends; a female, first Luer connector on the proximal end of the housing, having a first lumen defined therein; a male, second Luer connector on the distal end of the housing, a male Luer tip thereof having a second lumen defined therein; and a first passage formed in the housing, in fluid communication with the first and second lumens;
inserting a hollow sleeve into the first passage, with a proximal axial end thereof projecting out of the first passage and the first lumen towards the proximal axial end of the housing;
coupling the female, first Luer connector to the barrel Luer connector, with the proximal axial end of the hollow sleeve inserted within the outlet of the male, barrel Luer tip;
coupling a selectively removeable storage cap to the male, second Luer connector of the syringe coupler;
pre-filling the syringe cavity with a flushing solution; and
inserting a translatable syringe plunger into the syringe barrel within the syringe cavity.

12. The method for manufacturing the flush syringe of claim 11, further comprising sterilizing a pre-filled flush syringe after insertion of the syringe plunger; and packaging the now sterilized, pre-filled flush syringe in a sterile packaging container.

13. A method flushing a patient port during intravenous fluid administration to a patient by using the flush syringe of claim 1, comprising:
disinfecting a hub of a patient port having a hub Luer connector;
coupling the male, second Luer connector of the syringe coupler to the hub Luer connector of the patient port;
dispensing a first portion of pre-filled flushing solution retained within the syringe cavity through the male, second Luer connector into the patient port by depressing the syringe plunger;
de-coupling the male, second Luer connector from the hub Luer connector;
disinfecting the hub of the patient port;
administering one or more drugs through the hub with drug dosing syringes, disinfecting the hub after delivery of each drug;
decoupling female, first Luer connector of the syringe coupler, including the hollow sleeve, from the male, barrel Luer connector of the syringe, thereafter, discarding the syringe coupler and its hollow sleeve;
disinfecting the hub of the patient port;
coupling the male, barrel Luer connector to the hub Luer connector of the patient port and dispensing a second portion of the pre-filled flushing solution retained with the syringe cavity through the male, barrel Luer connector into the patient port by depressing the syringe plunger;
decoupling the male, barrel Luer connector from the hub Luer connector of the patient port, thereafter, discarding the syringe;
disinfecting the hub of the patient port; and
capping the hub of the patient port with a sterilized hub cap.

14. The method for flushing a patient port of claim 13, further comprising: removing the flush syringe from sterile packaging and decoupling a storage cap from the male, second Luer connector of the syringe coupler, prior to coupling latter to the hub Luer connector.

15. The method for flushing a patient port of claim 14, further comprising initially disinfecting the Luer hub of the patient port with a scrubbing cap assembly that is pre-coupled to the storage cap, within the sterile packaging of the flush syringe, prior to coupling the male, second Luer connector of the syringe coupler to the hub Luer connector.

16. A syringe coupler for intravenous fluid administration to a patient, comprising:
a housing having proximal and distal axial ends;
a female, first Luer connector on the proximal end of the housing, having a first lumen defined therein;
a male, second Luer connector on the distal end of the housing, a male Luer tip thereof having a second lumen defined therein;
a first passage formed in the housing, in fluid communication with the first and second lumens;
a hollow sleeve retained within the first passage, a proximal axial end of the hollow sleeve projecting out of the first passage and the first lumen towards the proximal axial end of the housing;
the female first Luer connector of the housing adapted for coupled receipt of a male Luer tip of a Luer connector of a flush syringe, whereby an outlet lumen formed within the syringe's male Luer tip receives the proximal axial end of the hollow sleeve therein for delivery of flushing solution from the flush syringe through the hollow sleeve, into the second lumen of the housing's male, second Luer connector; and
the male, second Luer connector of the housing adapted for coupled insertion into a corresponding needle-free IV connector of a patient, for delivery of flushing solution therefrom into the IV connector.

17. The syringe coupler of claim 16, further comprising a threaded male, second Luer connector on the distal end of the housing.

18. The syringe coupler of claim 16, the housing further comprising a coupling sleeve defining an internal coupling cavity on a proximal axial end thereof for slidable receipt of an exterior circumferential surface of an outer collar of the corresponding male, Luer connector of a flush syringe, and a distal axial end of the coupling sleeve coupled to the proximal, axial end of the housing.

19. The syringe coupler of claim 18, further comprising the distal axial end of the coupling sleeve and the proximal axial end of the housing selectively joined by a selectively engageable snap fitting, which when engaged, prevents axial separation of the coupling sleeve and the corresponding male, barrel Luer connector.

20. The syringe coupler of claim 19, the snap fitting comprising a radially projecting circumferential flange oriented on the coupling sleeve or the housing and opposing, mating, biased hooks on the other of the coupling sleeve or the housing that engage the circumferential flange and prevent axial separation of the coupling sleeve and the corresponding male, barrel Luer connector when so engaged.

21. A kit comprising:
one or more syringe couplers; and
a single flush syringe to be used for multiple fresh tip connections to flush a line between multiple or subsequent drug deliveries.
